(19) United States
(12) Patent Application Publication
(10) Pub. No.: US 2026/0167926 A1
(43) Pub. Date: Jun. 18, 2026

(54) SPINAL CORD INJURY TREATMENT NEUROSPHERE

(71) Applicant: KEIO UNIVERSITY, Tokyo (JP)

(72) Inventors: Hideyuki OKANO, Tokyo (JP); Masaya NAKAMURA, Tokyo (JP); Yoshitaka KASE, Tokyo (JP); Toshiki OKUBO, Tokyo (JP)

(21) Appl. No.: 19/532,578

(22) Filed: Feb. 6, 2026

Related U.S. Application Data
(62) Division of application No. 17/105,789, filed on Nov. 27, 2020.

(30) Foreign Application Priority Data
Nov. 28, 2019 (JP) 2019-215161

(51) Int. Cl. C12N 5/0793; A61K 35/30; A61P 25/00; C12N 5/074

(52) U.S. Cl. CPC C12N 5/0619; A61K 35/30; A61P 25/00; C12N 5/0607

(57) ABSTRACT

A neurosphere for spinal cord injury treatment in which the phosphorylation of p38 MAPK is enhanced as compared to the control, where the control is a neurosphere that has not contacted with a γ-secretase inhibitor, or neurons obtained by inducing differentiation from a neurosphere that has not contacted with a γ-secretase inhibitor, is useful for the treatment of spinal cord injuries.

### BACKGROUND OF THE INVENTION

### Field of the Invention

The present invention relates to a neurosphere inducer for spinal cord injury treatment and use of the neurosphere inducer. More particularly, the present invention relates to a neurosphere inducer for treating spinal cord injury, a neurosphere for treating spinal cord injury, and a method for screening a neurosphere inducer for spinal cord injury treatment. This application is a divisional of U.S. patent application Ser. No. 17/105,789, filed Nov. 27, 2020, which claims priority to Japanese Patent Application No. 2019-215161, filed in Japan on Nov. 28, 2019, the contents of each which are incorporated herein by reference.

### Description of Related Art

Spinal cord injuries are catastrophic injuries and cause paralysis, sensory impairment, neuropathic pain and bowel-bladder dysfunction. A large number of studies have reported on transplanting neural stem cells or neural progenitor cells for the treatment of spinal cord injuries; however, the optimal time frame for transplantation is considered to be the subacute phase after a spinal cord injury. Further, it is considered difficult to treat spinal cord injuries in the chronic phase due to changes in the intramedullary environment such as glial scarring and formation of cavities (see, for example, Keirstead H. S., et al., Human Embryonic Stem Cell-Derived Oligodendrocyte Progenitor Cell Transplants Remyelinate and Restore Locomotion after Spinal Cord Injury, J. Neurosci., 25(19 ), 4694-4705, 2005). However, since most patients with spinal cord injuries are in the chronic phase, there is a demand to develop a therapeutic technique that is applicable even to spinal cord injuries in the chronic phase.

### SUMMARY OF THE INVENTION

An object of the present invention is to provide a technology for treating spinal cord injuries.

The present invention includes the following aspects.
[1] A neurosphere inducer for spinal cord injury treatment, the neurosphere inducer containing a γ-secretase inhibitor as an active ingredient.
[2] A neurosphere for spinal cord injury treatment, in which phosphorylation of p38 MAPK is enhanced as compared to a control.
[3]The neurosphere for spinal cord injury treatment according to [2], which is produced by culturing in presence of a γ-secretase inhibitor.
[4]A method for producing the neurosphere for spinal cord injury treatment according to [2], the method including a step of culturing a pluripotent stem cell-derived neurosphere in presence of a γ-secretase inhibitor.
[5]A method for screening a neurosphere inducer for spinal cord injury treatment, the method including: a step of inducing differentiation of a neurosphere into neurons, in which at least a part of the step is carried out in presence of a test substance; a step of measuring phosphorylation of p38 MAPK in the induced neurons; and a step of selecting the test substance as a neurosphere inducer for spinal cord injury treatment in a case where phosphorylation of p38 MAPK thus measured has been enhanced as compared to the control.

A method for screening a neurosphere inducer for spinal cord injury treatment, the method including: a culturing step of culturing a neurosphere in presence of a test substance, a step of measuring phosphorylation of p38 MAPK in the neurosphere after the culturing step; and a step of selecting the test substance as a neurosphere inducer for spinal cord injury treatment in a case where phosphorylation of p38 MAPK thus measured has been enhanced as compared to the control.

According to the present invention, a technology for treating spinal cord injury can be provided.

### BRIEF DESCRIPTION OF THE DRAWINGS

FIG. 1A and FIG. 1B are graphs showing the results of quantitatively determining photons released from transplanted cells by bioluminescence imaging in Experimental Example 1.

FIG. 2A is representative fluorescence microscopic images showing the results of immunostaining in Experimental Example 1. FIG. 2B is a graph obtained by quantifying the results of FIG. 2A.

FIG. 3A is representative fluorescence microscopic images showing the results of immunostaining in Experimental Example 1. FIG. 3B is a graph obtained by quantifying the results of FIG. 3A.

FIG. 4A is representative microscopic images showing the results of hematoxylin/eosin staining in Experimental Example 2. FIG. 4B is a graph obtained by quantifying the results of FIG. 4A.

FIG. 5A is representative microscopic images showing the results of hematoxylin/eosin staining in Experimental Example 2. FIG. 5B is a graph obtained by quantifying the results of FIG. 5A.

FIG. 6A is representative microscopic images showing the results of Luxol Fast Blue (LFB) staining in Experimental Example 2. FIG. 6B is a graph obtained by qquantifying the results of FIG. 6A.

FIG. 7A is representative microscopic images showing the results of LFB staining in Experimental Example 2. FIG. 7B is a graph obtained by quantifying the results of FIG. 7A.

FIG. 8A is representative microscopic images showing the results of immunostaining in Experimental Example 3. FIG. 8B is a graph obtained by quantifying the results of FIG. 8A.

FIG. 9A is representative microscopic iamges showing the results of immunostaining in Experimental Example 3. FIG. 9B is a graph obtained by quantifying the results of FIG. 9A.

FIG. 10A is representative microscopic image showing the results of immunostaining in Experimental Example 3. FIG. 10B is a graph obtained by quantifying the results of FIG. 10A.

FIG. 11A is representative microscopic images showing the results of immunostaining in Experimental Example 3. FIG. 11B is a graph obtained by quantifying the results of FIG. 11A.

FIG. 12A and FIG. 12B are representative images showing the results of immunoelectron microscopy in Experimental Example 3.

FIG. 13A and FIG. 13B are graphs showing the results of measuring the length of neurites in Experimental Example 4.

FIG. 14A and FIG. 14B are representative microscopic images showing the results of immunostaining in Experimental Example 4.

FIG. 15A shows images of the results of Western blotting in Experimental Example 4. FIG. 15B is a graph showing the results of quantitatively determining non-phosphorylated p38 in Experimental Example 4. FIG. 15C is a graph showing the results of quantitatively determining phosphorylated p38 in Experimental Example 4.

FIG. 16A and FIG. 16B are representative microscopic images showing the results of immunostaining in Experimental Example 4. FIG. 16C is a graph showing the results of calculating the proportions of phosphorylated p38 positive cells in the transplanted cells based on FIG. 16A and FIG. 16B.

FIG. 17A and FIG. 17B are representative microscopic images showing the results of immunostaining in Experimental Example 5.

FIG. 18A and FIG. 18B are representative microscopic images showing the results of immunostaining in Experimental Example 5.

FIG. 19A and FIG. 19B are representative microscopic images showing the results of immunostaining in Experimental Example 5.

FIG. 20A and FIG. 20B are representative images showing the results of immunoelectron microscopy in Experimental Example 5.

FIG. 21A and FIG. 21B are representative microscopic images of spinal cord sections with which the reticulospinal tract stained with biotin dextran amine (BDA) was observed in Experimental Example 6. FIG. 21C is a graph showing the results of measuring the relative value of the proportion of the area of BDA-stained reticulospinal tract fibers at various positions covering from a position 4 mm away toward the rostral side of the site of a spinal cord injury extending to a position 4 mm away toward the caudal side of the site of a spinal cord injury.

FIG. 22A and FIG. 22B are graphs showing the results of measuring the Basso Mouse Scale (BMS) scores in Experimental Example 7.

FIG. 23A to FIG. 23D are graphs showing the results of evaluating the walking function in Experimental Example 7.

FIG. 24A and FIG. 24B are graphs showing the results of a rotarod test in Experimental Example 7.

FIG. 25 is a graph showing the relationship between the BMS score and the proportion of the area of reticulospinal tract fibers in Experimental Example 7.

FIG. 26 is a representative microscopic images showing the results of immunostaining in Experimental Example 8.

### DETAILED DESCRIPTION OF THE INVENTION

### Neurosphere Inducer for Spinal Cord Injury Treatment

According to an embodiment, the present invention provides a neurosphere inducer for spinal cord injury treatment, the neurosphere containing a γ-secretase inhibitor as an active ingredient.

According to the present specification, a neurosphere means a spherical cell aggregate formed by suspension growth culture of neural stem cells or neural progenitor cells. A neural stem cell means a cell having a capacity for self-replication and a capacity for differentiation into a neural progenitor cell. Furthermore, a neural progenitor cell is a cell that is undifferentiated but has been one-step differentiated from a neural stem cell, and is a cell that undergoes self-proliferation of the cell itself and finally differentiates into a neuron.

It is preferable that the neural stem cells or neural progenitor cells are preferably obtained by inducing differentiation from pluripotent stem cells. Examples of the pluripotent stem cells include embryonic stem cells (ES cells) and induced pluripotent stem cells (iPS cells). It is preferable that the pluripotent stem cells are human cells.

The method for inducing differentiation of pluripotent stem cells into neural stem cells or neural progenitor cells is not particularly limited and may be a method that is usually carried out. For example, a neurosphere can be obtained by subjecting pluripotent stem cells to suspension culture in a medium containing at least one of Basic fibroblast growth factor (bFGF) or Epidermal Growth Factor (EGF). Furthermore, a process of dissociating the neurosphere thus obtained into single cells, subjecting the single cells to suspension culture in a medium containing bFGF again, and allowing the cells to form a neurosphere again, may be repeated a plurality of times.

As will be described later in the Examples, spinal cord injuries can be treated by allowing a γ-secretase inhibitor to act on the neurosphere and transplanting the neurosphere into a site of a spinal cord injury of a chronic phase spinal cord injury model mouse. Therefore, it can be said that the γ-secretase inhibitor is a neurosphere inducer for spinal cord injury treatment.

Examples of the γ-secretase inhibitor include DAPT (CAS Number: 208255-80-5) and Comound 34 (CAS Number: 564462-36-8).

It is preferable that the γ-secretase inhibitor is caused to act at the highest concentration at which the γ-secretase inhibitor effectively suppresses cell division of a neurosphere when added to the medium of the neurosphere, does not exhibit noticeable cytotoxicity to the neurosphere, and does not form a precipitate in the medium. For example, in a case where DAPT is used as the γ-secretase inhibitor, the final concentration of DAPT in the medium of the neurosphere is preferably 10 to 1000 μM, more preferably 10 to 100 μM, and even more preferably 10 μM.

The period for allowing the γ-secretase inhibitor to act on the neurosphere is preferably 20 to 24 hours.

### Neurosphere for Spinal Cord Injury Treatment

According to an embodiment, the invention provides a neurosphere for spinal cord injury treatment, in which phosphorylation of p38 MAPK is significantly enhanced as compared to the control.

As will be described later in Examples, spinal cord injuries can be treated by transplanting the neurosphere of the present embodiment into a site of a spinal cord injury of a chronic phase spinal cord injury model mouse. The inventors also revealed that the phosphorylation of p38 MAPK is enhanced in a neurosphere that has been cultured in the presence of a γ-secretase inhibitor. In addition, the inventors have revealed that the phosphorylation of p38 MAPK is enhanced in the neurons obtained by inducing differentiation from the neurosphere of the present embodiment.

In the neurosphere of the present embodiment, the control may be a neurosphere that has not contacted with a γ-secretase inhibitor, or neurons obtained by inducing differentiation from a neurosphere that has not contacted with a γ-secretase inhibitor.

Phosphorylation of p38 MAPK can be measured by immunostaining using an antibody specific to phosphorylated p38, Western blotting, or the like.

It is preferable that the neurosphere of the present embodiment is cells produced by being cultured in the presence of a γ-secretase inhibitor. The γ-secretase inhibitor and the neurosphere are as described above.

### Method for Producing Neurosphere for Spinal Cord Injury Treatment

According to an embodiment, the present invention provides a method for producing a neurosphere for spinal cord injury treatment, the method including a step of culturing a pluripotent stem cell-derived neurosphere in the presence of a γ-secretase inhibitor. The above-mentioned neurosphere for spinal cord injury treatment can be produced by the production method of the present embodiment. With regard to the production method of the present embodiment, the pluripotent stem cells, the neurosphere, and the γ-secretase inhibitor are as described above.

### Method for Screening Neurosphere Inducer for Spinal Cord Injury Treatment

### First Embodiment

The screening method of the first embodiment is a method for screening a neurosphere inducer for spinal cord injury treatment, the method including a step of inducing differentiation of a neurosphere into neurons, in which at least a part of the step is carried out in the presence of a test substance; a step of measuring phosphorylation of p38 MAPK of the induced neurons; and a step of selecting the test substance as a neurosphere inducer for spinal cord injury treatment when the phosphorylation of p38 MAPK thus measured is significantly enhanced as compared to the control.

As will be described later in the Examples, the inventors found that in the neurons obtained by inducing differentiation of a neurosphere that is capable of treating spinal cord injuries in the chronic phase by being transplanted into a site of a spinal cord injury, phosphorylation of p38 MAPK is enhanced as compared to the control. Therefore, it can be said that a test substance that enhances the phosphorylation of p38 MAPK by acting on cells in at least a part of a step of inducing differentiation of a neurosphere into neurons, is a neurosphere inducer for spinal cord injury treatment.

The test substance may act on cells in at least a part of a step of inducing differentiation of a neurosphere into neurons or may act on cells in the entire step of inducing differentiation of a neurosphere into neurons.

For example, the test substance may be allowed to act for 8 to 72 hours, may be allowed to act for 12 to 48 hours, or may be allowed to act for 20 to 24 hours.

As the control, neurons obtained by inducing differentiation from a neurosphere that has not contacted with a γ-secretase inhibitor, may be mentioned.

The test substance is not particularly limited, and examples include a natural compound library, a synthetic compound library, an existing drug library, and a metabolite library.

In addition, the phosphorylation of p38 MAPK can be measured by immunostaining using an antibody specific to phosphorylated p38, Western blotting, or the like.

### Second Embodiment

The screening method of the second embodiment is a method for screening a neurosphere inducer for spinal cord injury treatment, the method including a step of culturing a neurosphere in the presence of a test substance, a step of measuring phosphorylation of p38 MAPK in the neurosphere after culturing, and a step of selecting the test substance as a neurosphere inducer for spinal cord injury treatment in a case where the phosphorylation of p38 MAPK thus measured is significantly enhanced as compared to the control.

In the screening method of the first embodiment, the phosphorylation of p38 MAPK is measured after a neurosphere that has been cultured in the presence of the test substance is induced to differentiate into neurons, whereas the screening method of the second embodiment is mainly different in that the phosphorylation of p38 MAPK is measured without inducing differentiation of a neurosphere that has been cultured in the presence of the test substance into neurons. A neurosphere inducer for spinal cord injury treatment can also be screened by the screening method of the second embodiment.

In the screening method of the second embodiment, the control may be a neurosphere that has not contacted with a γ-secretase inhibitor. Furthermore, the test substance and the method for measuring phosphorylation of p38 MAPK are similar to those of the screening method of the first embodiment.

### Other Embodiments

According to an embodiment, the present invention provides a method for treating spinal cord injuries, the method including a step of transplanting an effective amount of a neurosphere in which phosphorylation of p38 MAPK is enhanced, into an injured site of a patient with a spinal cord injury.

It is preferable that the neurosphere is cells whose rejection reaction against a patient is suppressed. The neurosphere in which phosphorylation of p38 MAPK has been enhanced is as described above.

### EXAMPLES

Next, the present invention will be described in more detail by disclosing Examples; however, the present invention is not intended to be limited to the following Examples. All animal experiments were conducted in accordance with the guidelines of the Laboratory Animal Committee, Keio University School of Medicine, and the guidelines of the National Institutes of Health (NIH) in the US (Keio University Approval No. 13020).

### Experimental Example 1

(Transplantation of Human iPS cell-derived Neurospheres Treated with γ-secretase Inhibitor into Chronic Phase Spinal Cord Injury Model Mouse—1)

### <<Production of a Spinal Cord Injury Model Mouse>>

NOD/ShiJic-scidJcI mice (8 weeks old, female), which are immune-deficient mice, were anesthetized. Subsequently, a laminectomy was performed on the 10th thoracic vertebra to expose the dorsal dura mater, and then spinal cord injury of a moderate degree caused by crush injury was formed. For the formation of a spinal cord injury, an IH impactor (manufactured by Precision Systems and Instrumentation LLC) equipped with a stainless steel tip was used, and an impact with a force set to 60 kdyn (0.6 N) was applied.

### <<Preparation of Neurospheres>>

Human iPS cell strains 201B7 and 414C2 were subjected to adherent culture for 12 days together with mouse embryo-derived fibroblasts. Subsequently, the cells were subjected to suspension culture for 30 days to form embryoid bodies. Subsequently, the aggregated cells were differentiated into neurospheres.

### <<Lentivirus Infection>>

The 201B7 cell-derived neurospheres and the 414C2 cell-derived neurospheres were respectively dissociated, and a construct that expresses ffLuc under the control of EF promoter was introduced using a lentivirus. ffLuc is a fusion protein of firefly luciferase and Venus fluorescent protein. Thereby, transplanted cells can be identified by detecting the signal of luciferase or the fluorescent signal of Venus.

Since the luciferin-luciferase reaction is ATP-dependent, only living cells release photons. Therefore, the survival of transplanted cells over time can be evaluated by a bioluminescence imaging (BLI) system.

<<Cell Transplantation into Spinal Cord Injury Model Mouse>>

DAPT (CAS Number: 208255-80-5, Sigma-Aldrich Corporation), which is a γ-secretase inhibitor, was dissolved in dimethyl sulfoxide (DMSO) to a final concentration of 10 mM and used.

Prior to transplantation, 201B7 cell-derived neurospheres and 414C2 cell-derived neurospheres were respectively cultured for one day in the presence of DAPT at a final concentration of 10 μM. Furthermore, a group cultured in the absence of DAPT (a group in which only the solvent in which DAPT was dissolved was added to the medium) was also prepared.

42 days after the spinal cord injury, DAPT-treated human iPS cell-derived neurospheres (hereinafter, may be referred to as “GSI (+) group”) or non-DAPT-treated human iPS cell-derived neurospheres (hereinafter, may be referred to as “GSI (−) group”) were transplanted into the central part of the injured site of each mouse at a cell density of 5×10⁵ cells/2 μL. The mice were grouped with n=10 in each group. Transplantation was performed at a rate of 1 μL/min using a glass micropipette and using a 25-μL syringe (manufactured by Hamilton Company) and a stereotaxic microinjector (product name “KDS 310”, manufactured by Muromachi Kikai Co., Ltd.). In addition, a group into which the same volume of phosphate buffered saline (PBS) was transplanted (hereinafter, may be referred to as “PBS group”) was also prepared.

### <<Evaluation of Survival Rate of Transplanted Cells>>

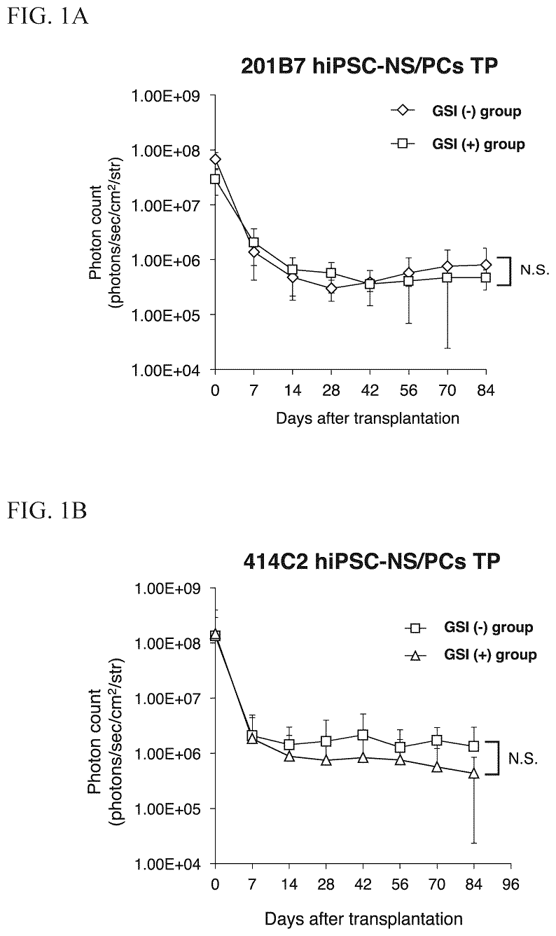

FIG. 1A and FIG. 1B are graphs showing the results of quantitatively determining the photons released from transplanted cells by bioluminescence imaging performed over time after cell transplantation (n=10). FIG. 1A shows the results of transplantation of 201B7 cell-derived neurospheres, and FIG. 1B shows the results of transplantation of 414C2 cell-derived neurospheres.

In FIG. 1A and FIG. 1B, the axis of abscissa represents the number of days since cell transplantation, and the axis of ordinate represents the number of detected photons. In addition, the term “GSI (+) group” indicates the results for the group into which DAPT-treated human iPS cell-derived neurospheres were transplanted, the term “GSI (−) group” indicates the results for the group into which non-DAPT-treated human iPS cell-derived neurospheres were transplanted, and the term “N.S.” indicates that there is no significant difference.

As a result, it was found that in both the case where a DAPT treatment was carried out and the case where a DAPT treatment was not carried out, the number of photons decreased for 7 days after transplantation and reached a plateau 14 days after transplantation, and thereafter, the number of photons was maintained until after 3 months from the transplantation.

From these results, it was found that both the DAPT-treated human iPS cell-derived neurospheres and the non-DAPT-treated human iPS cell-derived neurospheres continued to survive even in the chronic phase of a spinal cord injury after transplantation, and conspicuous oncogenicity was also not observed.

### <<Evaluation of Differentiation State of Transplanted Cells>>

In order to evaluate the differentiation state of the cells transplanted into the central part of an injured site, immunostaining of various cell markers was performed using spinal cord sections obtained 84 days after the transplantation, and a quantitative analysis was performed. Human nuclear antigen (HNA), Ki67, Nestin, pan-ELAVL (Hu), GFAP, and APC were immunostained as cell markers.

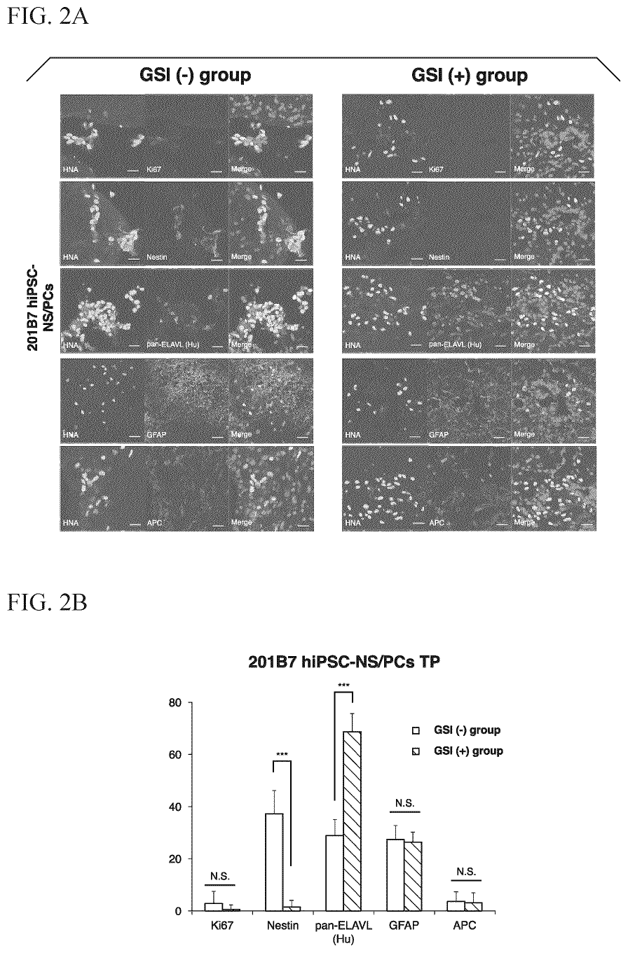

FIG. 2A and FIG. 2B show the results of immunostaining of tissues in which 201B7 cell-derived neurospheres were transplanted into a site of a spinal cord injury. FIG. 2A is representative fluorescence microscopic images showing the results of immunostaining, and FIG. 2B is a graph showing the proportions (%) of the numbers of various marker-positive cells in the HNA-positive cells of FIG. 2A (n=10).

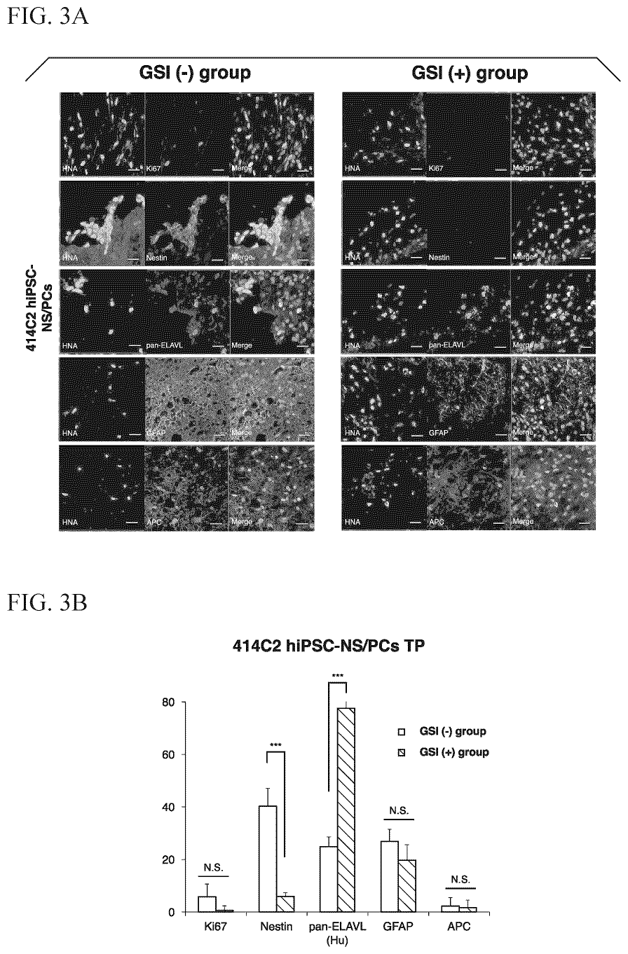

In addition, FIG. 3A and FIG. 3B show the results of immunostaining of tissues in which 414C2 cell-derived neurospheres were transplanted into a site of a spinal cord injury. FIG. 3A is representative fluorescence microscopic images showing the results of immunostaining, and FIG. 3B is a graph showing the proportions (%) of the numbers of various marker-positive cells in the HNA-positive cells of FIG. 3A (n=10).

In FIG. 2A, FIG. 2B, FIG. 3A, and FIG. 3B, the term “GSI (-) group” indicates the results for the group into which non-DAPT-treated human iPS cell-derived neurospheres were transplanted, and the term “GSI (+) group” indicates the results for the group into which DAPT-treated human iPS cell-derived neurospheres were transplanted. Furthermore, the axis of ordinate of FIG. 2B and FIG. 3B represents the proportions (%) of the numbers of various marker-positive cells in HNA-positive cells, the symbol “***” indicates that there is a significant difference at p<0.001, and the term “N.S.” indicates that there is no significant difference.

As a result, it was found that both the 201B7 cell-derived neurospheres and the 414C2 cell-derived neurospheres, both of which had been transplanted, were differentiated into three cell lineages of the nervous system, namely, pan-ELAVL (Hu)-positive mature neurons, GFAP-positive astrocytes, and APC-positive oligodendrocytes.

In addition, it was found that in the GSI (+) group, the proportion of pan-ELAVL (Hu)-positive mature neurons was significantly increased, and the proportion of Nestin-positive cells was significantly decreased as compared to the GSI (−) group. On the other hand, regarding the proportions of GFAP-positive astrocytes and APC-positive oligodendrocytes, there was no significant difference in the GSI (+) group and the GSI (−) group.

### Experimental Example 2

(Transplantation of Human iPS Cell-derived Neurospheres Treated with γ-secretase Inhibitor Into Chronic Phase Spinal Cord Injury Model mice—2)

In Experimental Example 1, spinal cord sections of each mouse obtained 84 days after transplantation were subjected to hematoxylin-eosin staining and Luxol Fast Blue (LFB) staining, the shapes of the spinal cord sections were observed, and the myelinated region was examined.

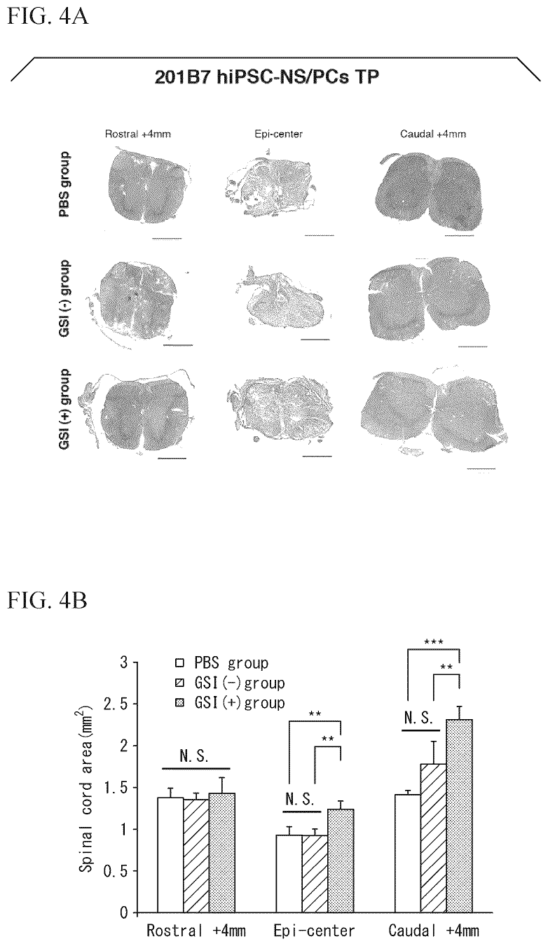

FIG. 4A and FIG. 4B show the results for mice into which 201B7 cell-derived neurospheres were transplanted. FIG. 4A is representative microscopic images showing the results of hematoxylin-eosin staining. The scale bar is 200 μm. FIG. 4B is a graph obtained by quantifying the results of FIG. 4A (n=10).

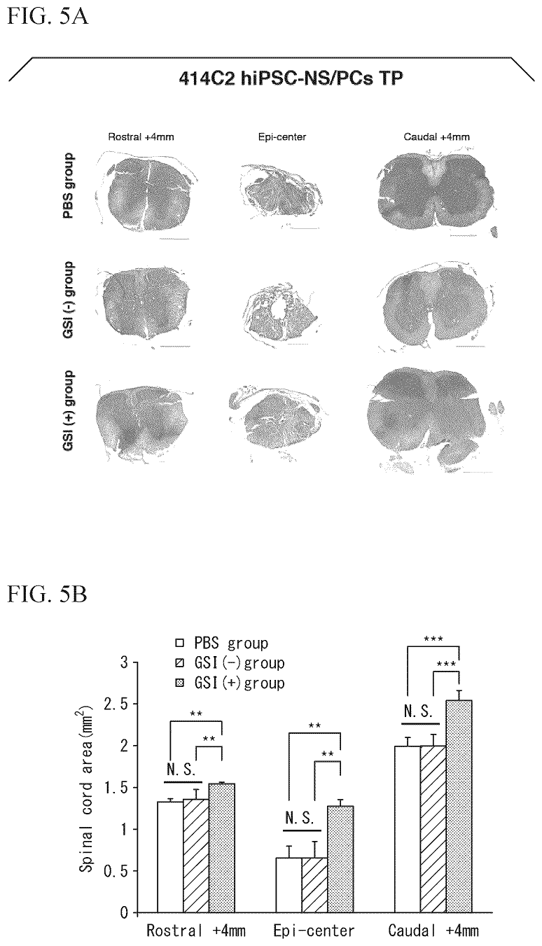

FIG. 5A and FIG. 5B show the results for mice into which 414C2 cell-derived neurospheres were transplanted. FIG. 5A is representative microscopic images showing the results of hematoxylin-eosin staining. The scale bar is 200 μm. FIG. 5B is a graph obtained by quantifying the results of FIG. 5A (n=10).

In FIG. 4A, FIG. 4B, FIG. 5A, and FIG. 5B, the term “PBS group” indicates the results for the group into which PBS was infused without transplanting cells, the term “GSI (−) group” indicates the results for the group into which non-DAPT-treated human iPS cell-derived neurospheres were transplanted, and the term “GSI (+) group” indicates the results for the group into which DAPT-treated human iPS cell-derived neurospheres were transplanted.

Furthermore, the term “Rostral +4 mm” indicates the results for a section of a site 4 mm away from the site of a spinal cord injury toward the rostral side, the term “Epi-center” indicates the results for a section of the site of a spinal cord injury, and the term “Caudal +4 mm” indicates the results for a section of a site 4 mm away from the site of a spinal cord injury toward the caudal side.

In addition, the axis of ordinate in FIG. 4B and FIG. 5B represents the area of the spinal cord, the symbol “***” indicates that there is a significant difference at p<0.001, the symbol “**” indicates that there is a significant difference at p<0.01, the symbol “*” indicates that there is a significant difference at p<0.05, and the term “N.S.” represents that there is no significant difference.

As a result of performing a quantitative analysis, it was found that the area of the spinal cord cross section at the site of a spinal cord injury and the area of the spinal cord cross section at the site 4 mm away from the site of a spinal cord injury toward the caudal side were significantly larger in the GSI (+) group as compared to the GSI (−) group and the PBS group.

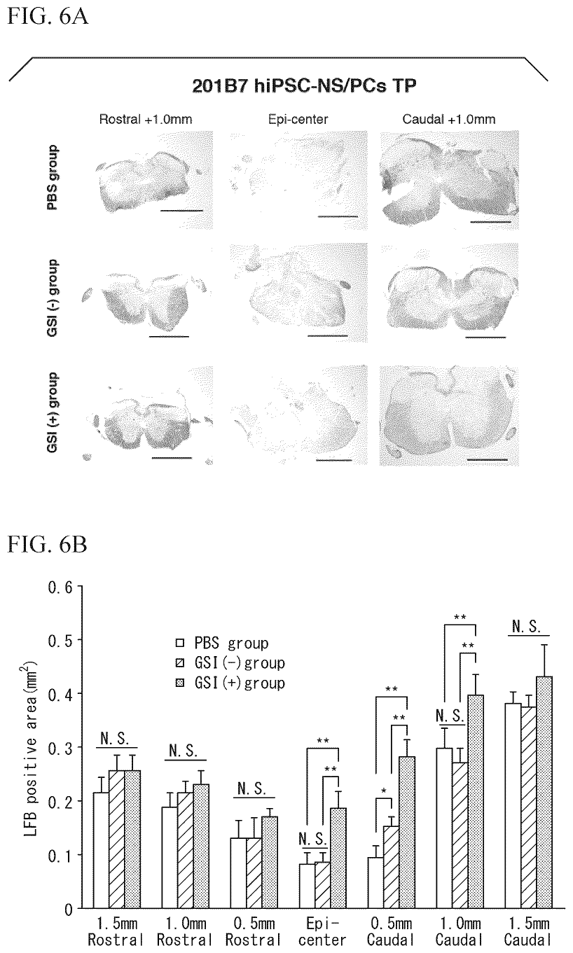

FIG. 6A and FIG. 6B show the results for the mice into which 201B7 cell-derived neurospheres were transplanted. FIG. 6A is representative microscopic images showing the results of LFB staining. The scale bar is 200 μm. FIG. 6B is a graph obtained by quantifying the results of FIG. 6A (n=10).

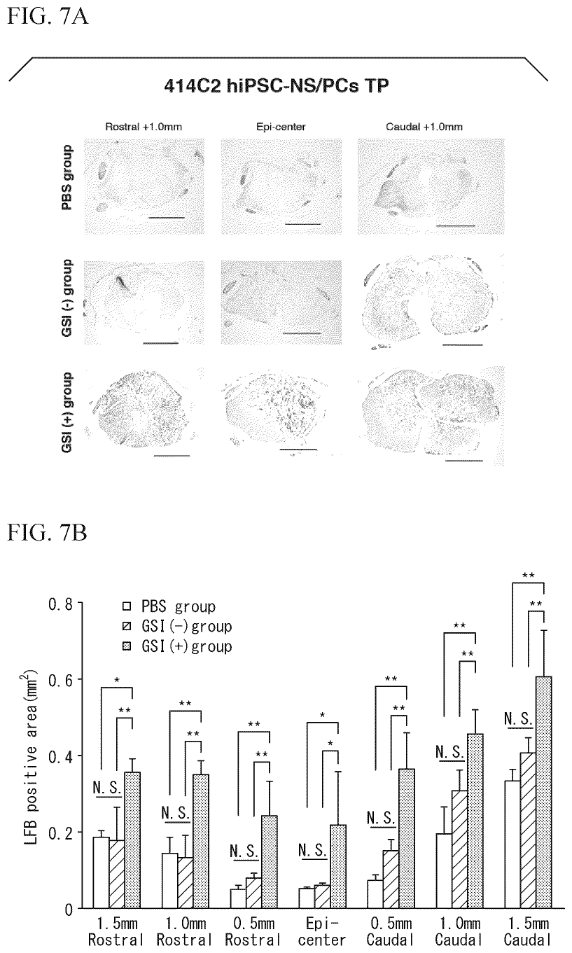

FIG. 7A and FIG. 7B show the results for the mice into which 414C2 cell-derived neurospheres were transplanted. FIG. 7A is representative microscopic images showing the results of LFB staining. The scale bar is 200 μm. FIG. 7B is a graph obtained by quantifying the results of FIG. 7A (n=10).

In FIG. 6A, FIG. 6B, FIG. 7A, and FIG. 7B, the term “PBS group” indicates the results for the group into which PBS was infused without transplanting cells, the term “GSI (−) group” indicates the results for the group into which non-DAPT-treated human iPS cell-derived neurospheres were transplanted, and the term “GSI (+) group” indicates the results for the group into which DAPT-treated human iPS cell-derived neurospheres were transplanted.

Furthermore, the term “Rostral +X mm” indicates the results for a section of a site X mm away from the site of a spinal cord injury toward the rostral side, the term “Epi-center” indicates the results for a section of the site of a spinal cord injury, and the term “Caudal +X mm” indicates the results for a section of a site X mm away from the site of a spinal cord injury toward the caudal side.

Furthermore, the axis of ordinate in FIG. 6B and FIG. 7B represents the area of the LFB staining positive region, the symbol “***” indicates that there is a significant difference at p<0.001, the symbol “**” indicates that there is a significant difference at p<0.01, the symbol “*” indicates that there is a significant difference at p<0.05, and the term “N.S.” indicates that there is no significant difference.

As a result of performing a quantitative analysis, it was found that in the GSI (+) group, the area of the LFB-staining-positive, myelinated region over a site 1 mm away from the site of a spinal cord injury toward the caudal side was significantly large as compared to the GSI (−) group and the PBS group.

These results show that human iPS cell-derived neurospheres that had been treated with a γ-secretase inhibitor led to remyelination of the site of a spinal cord injury in the chronic phase.

### Experimental Example 3

(Transplantation of Human iPS Cell-derived Neurospheres Treated with a γ-secretase Inhibitor Into Chronic Phase Spinal Cord Injury Model Mice—3)

The effect of axonal regeneration by transplantation of human iPS cell-derived neurospheres treated with a γ-secretase inhibitor was examined by immunostaining of spinal cord sections and immunoelectron microscopy. For immunostaining, neurofilament 200 kDa (NF-H) and 5-hydroxytryptamine (5HT) were stained.

### <<Immunostaining of NF-H>>

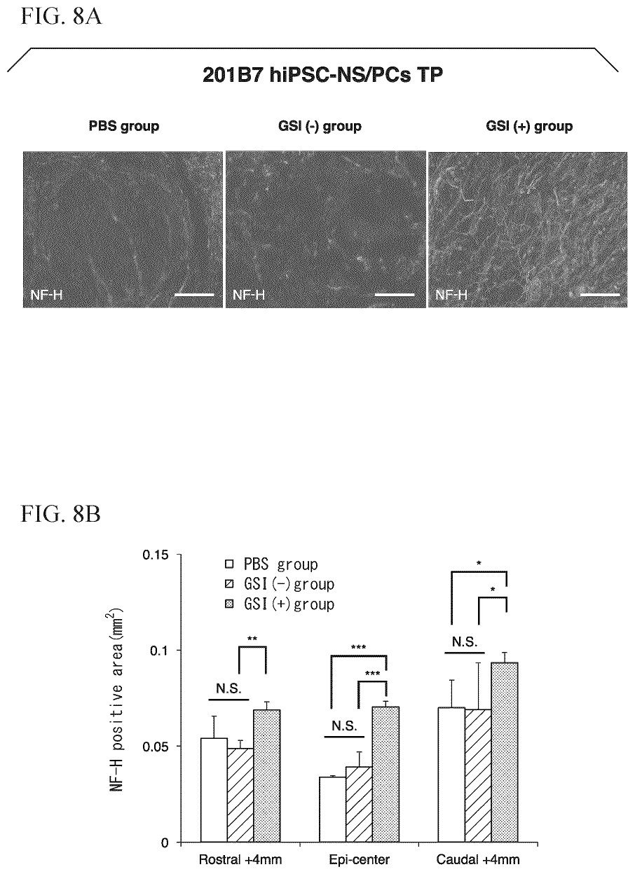

FIG. 8A and FIG. 8B show the results for the mice into which 201B7 cell-derived neurospheres were transplanted. FIG. 8A is representative microscopic images showing the results of immunostaining NF-H. The scale bar is 200 μm. FIG. 8B is a graph obtained by quantifying the results of FIG. 8A (n=10).

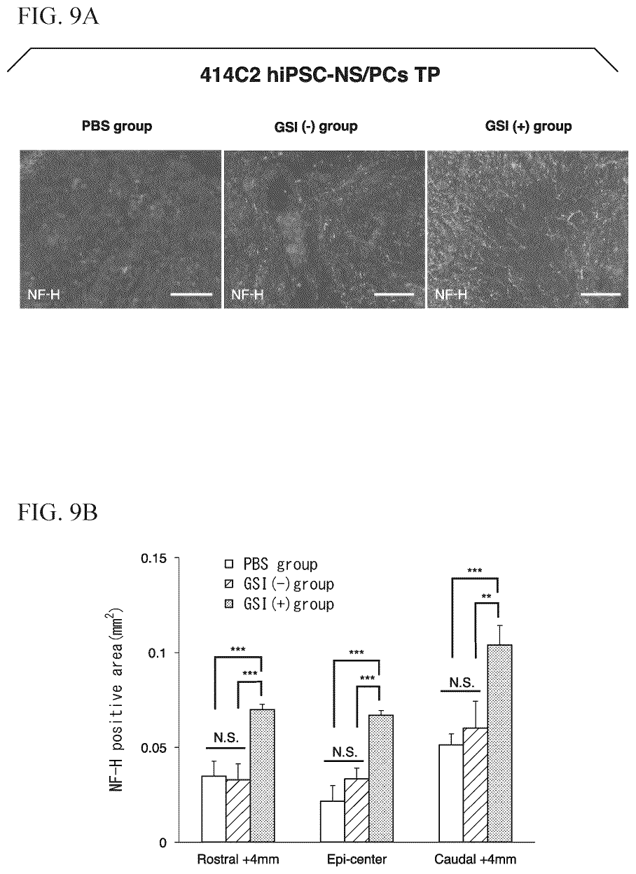

FIG. 9A and FIG. 9B are the results for the mice into which 414C2 cell-derived neurospheres were transplanted. FIG. 9A is representative microscopic images showing the results of immunostaining NF-H. The scale bar is 200 μm. FIG. 9B is a graph obtained by quantifying the results of FIG. 9A (n=10).

In FIG. 8A, FIG. 8B, FIG. 9A, and FIG. 9B, the term “PBS group” indicates the results for the group into which PBS was infused without transplanting cells, the term “GSI (−) group” indicates the results for the group into which non-DAPT-treated human iPS cell-derived neurospheres were transplanted, and the term “GSI (+) group” indicates the results for the group into which DAPT-treated human iPS cell-derived neurospheres were transplanted.

Furthermore, in FIG. 8B and FIG. 9B, the term “Rostral +4 mm” indicates the results for a section of a site 4 mm away from the site of a spinal cord injury toward the rostral side, the term “Epi-center” indicates the results for a section of the site of a spinal cord injury, and the term “Caudal +4 mm” indicates the results for a section of a site 4 mm away from the site of a spinal cord injury toward the caudal side.

The axis of ordinate in FIG. 8B and FIG. 9B represents the area of the NF-H-positive region, the symbol “***” indicates that there is a significant difference at p<0.001, the symbol “**” indicates that there is a significant difference at p<0.01, the symbol “*” indicates that there is a significant difference at p<0.05, and the term “N.S.” indicates that there is no significant difference.

As a result, with regard to the site of a spinal cord injury, a site 4 mm away from the site of a spinal cord injury toward the rostral side, and a site 4 mm away from the site of a spinal cord injury toward the caudal side, it was found that in the GSI (+) group, the number of NF-H-positive neuronal fibers was significantly large as compared to the GSI (−) group and the PBS group.

### <<Immunostaining of 5HT>>

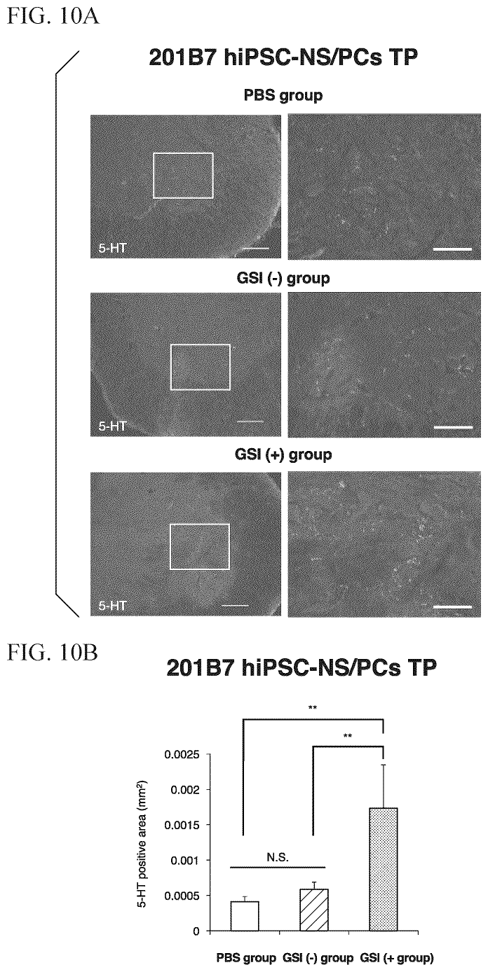

FIG. 10A and FIG. 10B show the results for the mice into which 201B7 cell-derived neurospheres were transplanted. FIG. 10A is representative microscopic images showing the results of immunostaining 5HT-positive serotonergic neuronal fibers in the lumbar intumescence. The scale bar is 200 μm. FIG. 10B is a graph obtained by quantifying the results of FIG. 10A (n=10).

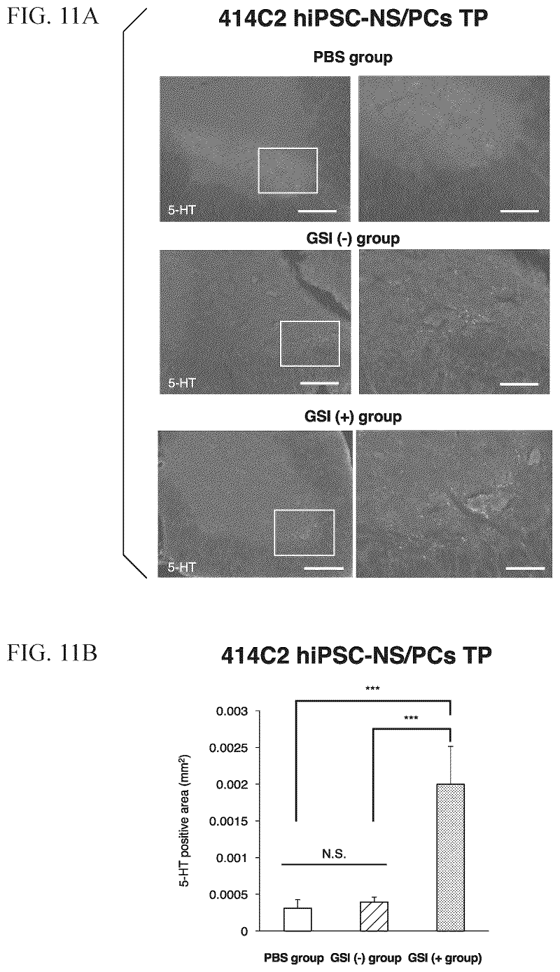

FIG. 11A and FIG. 11B show the results for the mice into which 414C2 cell-derived neurospheres were transplanted. FIG. 11A is representative microscopic images showing the results of immunostaining 5HT-positive serotonergic neuronal fibers in the lumbar intumescence. The scale bar is 200 μm. FIG. 11B is a graph obtained by quantifying the results of FIG. 11A (n=10).

In FIG. 10A, FIG. 10B, FIG. 11A, and FIG. 11B, the term “PBS group” indicates the results for the group into which PBS was infused without transplanting cells, the term “GSI (−) group” indicates the results for the group into which non-DAPT-treated human iPS cell-derived neurospheres were transplanted, and the term “GSI (+) group” indicates the results for the group into which DAPT-treated human iPS cell-derived neurospheres were transplanted.

The axis of ordinate in FIG. 10B and FIG. 11B represents the area of the 5HT-positive region, the symbol “***” indicates that there is a significant difference at p<0.001, the symbol “**” indicates that there is a significant difference at p<0.01, and the term “N.S.” indicates that there is no significant difference.

As a result, it was found that the number of 5HT-positive serotonergic neuronal fibers in the GSI (+) group was significantly large as compared to the GSI (−) group and the PBS group.

### <<Immunoelectron Microscopy>>

Frozen sections of the spinal cord of mice into which human iPS cell-derived neurospheres treated with a γ-secretase inhibitor had been transplanted were blocked, and then anti-human cytoplasmic antibody (STEM121™, mouse IgG1) was reacted with the sections. Subsequently, a nanogold-labeled goat anti-mouse antibody was reacted with the sections.

Subsequently, the sample was fixed with 2.5% glutaraldehyde, and the signal of nanogold was enhanced using a commercially available kit (product name “R-Gent SE-EM Silver Enhancement Reagents”, Aurion Immuno Gold Reagents & Accessoires). Subsequently, the sample was fixed with 1% OsO₄, dehydrated, and embedded in Epon. Subsequently, sliced sections (70 nm) were prepared using an ultramicrotome (product name “UC7”, manufactured by Leica Biosystems Nussloch GmbH), stained with uranium acetate and lead citrate, and observed using a transmission electron microscope (product name “Model 1400 Plus”, manufactured by JEOL, Ltd.).

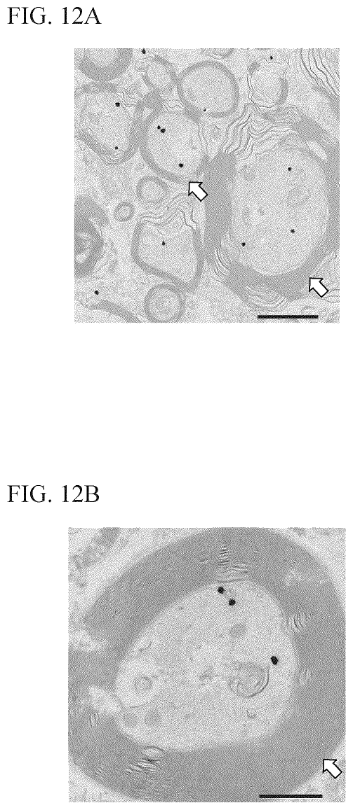

FIG. 12A and FIG. 12B are representative images showing the results of immunoelectron microscopy. The scale bar in FIG. 12A is 2 μm, and the scale bar in FIG. 12B is 200 nm. In FIG. 12A and FIG. 12B, the arrows indicate myelin lamella.

As a result, it was found that in chronic phase spinal cord injuries, a large number of regenerated axons derived from transplanted cells are actively remyelinated by host-derived glial cells.

### Experimental Example 4

(Analysis of Human iPS Cell-derived Neurospheres Treated with γ-secretase Inhibitor—1)

### <<Analysis on Cytokines>>

An examination was conducted on the possibility that human iPS cell-derived neurospheres that had been treated with a γ-secretase inhibitor may secrete some sort of neurotrophic factors as compared to human iPS cell-derived neurospheres that have not been treated with a γ-secretase inhibitor.

Specifically, using an anti-cytokine antibody array (manufactured by RayBiotech, Inc.), the expression levels of BDNF, b-NGF, HGF, CTNF, GDNF, NT-4, NT-3, PDGF-AA, and VEGF, all of which were secreted by human iPS cell-derived neurospheres that had been treated with a γ-secretase inhibitor and human iPS cell-derived neurospheres that had not been treated with a γ-secretase inhibitor, were quantitatively determined.

As a result, a factor whose expression level was conspicuously increased by the human iPS cell-derived neurospheres that had been treated with a γ-secretase inhibitor, as compared to the human iPS cell-derived neurospheres that had not been treated with a γ-secretase inhibitor, was not detected.

### <<Examination of p38 Phosphorylation>>

An examination was conducted on the phosphorylation of p38 MAPK as a mechanism by which human iPS cell-derived neurospheres treated with a γ-secretase inhibitor induce axonal regeneration in chronic phase spinal cord injuries.

Specifically, in an in vitro experimental system, human iPS cell-derived neurospheres were treated with a γ-secretase inhibitor and a p38 MAPK inhibitor to induce differentiation into neurons. Subsequently, the length of the neurite was measured and evaluated.

DAPT was used as the γ-secretase inhibitor. In addition, SB203580 (CAS Number: 152121-47-6) was used as the p38 MAPK inhibitor. First, human iPS cell-derived neurospheres were cultured for one day in the presence of the γ-secretase inhibitor alone, in the presence of the p38 MAPK inhibitor alone, or in the presence of the γ-secretase inhibitor and the p38 MAPK inhibitor.

Subsequently, various cells were seeded on a poly-L-ornithine/fibronectin-coated 48-well chamber slide (manufactured by Costar Corporation) at a cell density of 1×10⁵ cells/mL, and the cells were cultured in a growth factor-free medium at in a 95% air environment at 37° C. and 5% CO₂ for 14 days and were differentiated into neurons. Subsequently, on the 7th day and the 14th day after the initiation of differentiation induction, the cells were fixed with 4% paraformaldehyde in 0.1 M PBS, and the length of the neurite was measured.

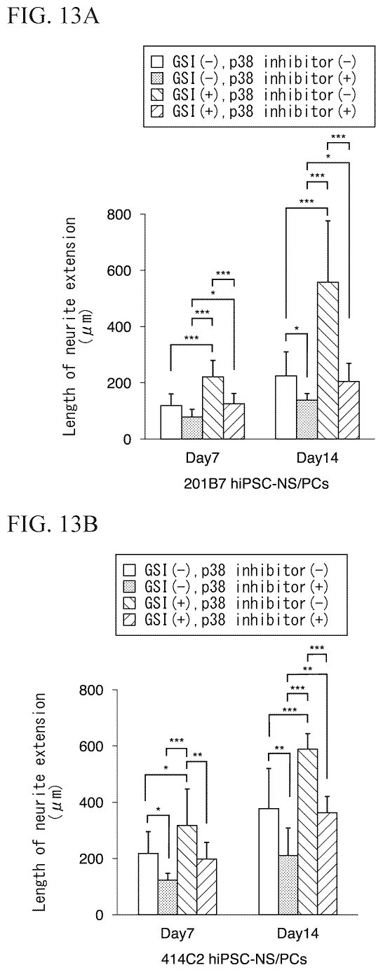

FIG. 13A is a graph showing the results for neurons derived from 201B7 cells (n=5). In addition, FIG. 13B is a graph showing the results for neurons derived from 414C2 cells (n=5). In FIG. 13A and FIG. 13B, “GSI” indicates DAPT, “p38 inhibitor” indicates SB203580, the symbol “(−)” indicates that the item was not added, and the symbol “(+)” indicates that the item was added. In addition, the axis of ordinate represents the length of the neurite, the symbol “***” indicates that there is a significant difference at p<0.001, the symbol “**” indicates that there is a significant difference at p<0.01, and the symbol “*” indicates that there is a significant difference at p<0.05.

As a result, it was found that when the human iPS cell-derived neurospheres were treated with a γ-secretase inhibitor, neurite outgrowth was significantly large as compared to the untreated group. In contrast, it was found that when the human iPS cell-derived neurospheres were treated with a p38 MAPK inhibitor, the neurite outgrowth was significantly decreased.

It was also found that when the human iPS cell-derived neurospheres were treated with both a γ-secretase inhibitor and a p38 MAPK inhibitor, the neurite outgrowth was equivalent to that of the untreated group.

Immunostaining of p38 MAPK, phosphorylated p38 MAPK, and βIII-tubulin was performed using cells obtained 14 days after the initiation of differentiation induction. In addition, Hoechst 33342 (CAS Number: 23491-52-3) was used to stain the cell nuclei.

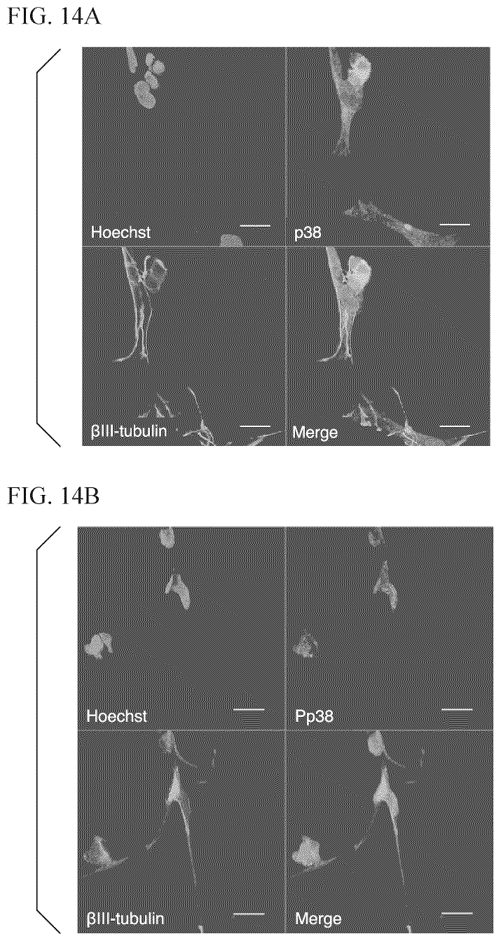

FIG. 14A and FIG. 14B are representative microscopic images showing the results of immunostaining of the cells in the GSI (+) group. In FIG. 14A and FIG. 14B, “Pp38” indicates phosphorylated p38 MAPK, and “Merge” indicates the results of superimposing microscopic images. The scale bar is 20μm.

As a result, it was found that in the cells treated with a γ-secretase inhibitor, βIII-tubulin-positive neurons and a p38-positive region were co-localized. Furthermore, a phosphorylated p38-positive region was in the nucleus.

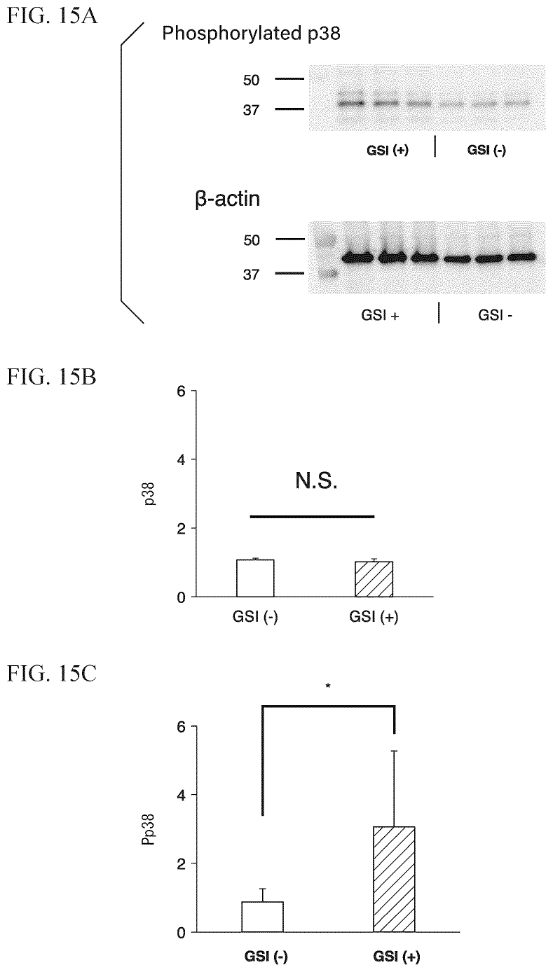

Subsequently, non-phosphorylated p38 and phosphorylated p38 were quantitatively determined by Western blotting. FIG. 15A is images showing the results of Western blotting. Western blotting of β-actin was performed as a loading control. FIG. 15B is a graph showing the results of quantitative determination of non-phosphorylated p38 MAPK. FIG. 15C is a graph showing the results of quantitative determination of phosphorylated p38 MAPK (Pp38).

In FIG. 15A to FIG. 15C, the term “GSI (+)” indicates the results for DAPT-treated human iPS cell-derived neurospheres, and the term “GSI (−)” indicates the results for non-DAPT-treated human iPS cell-derived neurospheres. Furthermore, the symbol “*” indicates that there is a significant difference at p<0.05, and the term “N.S.” indicates that there is no significant difference.

As a result, it was found that the amount of non-phosphorylated p38 was of the same extent between the GSI (−) group and the GSI (+) group, whereas the phosphorylated p38 was up-regulated in the GSI (+) group.

Subsequently, immunostaining of anti-human cytoplasmic antibody (STEM121™) and phosphorylated p38 MAPK was performed using spinal cord sections obtained 84 days after transplantation of human iPS cell-derived neurospheres into the above-mentioned chronic phase spinal cord injury model mice. Furthermore, Hoechst 33342 was used to stain the cell nuclei.

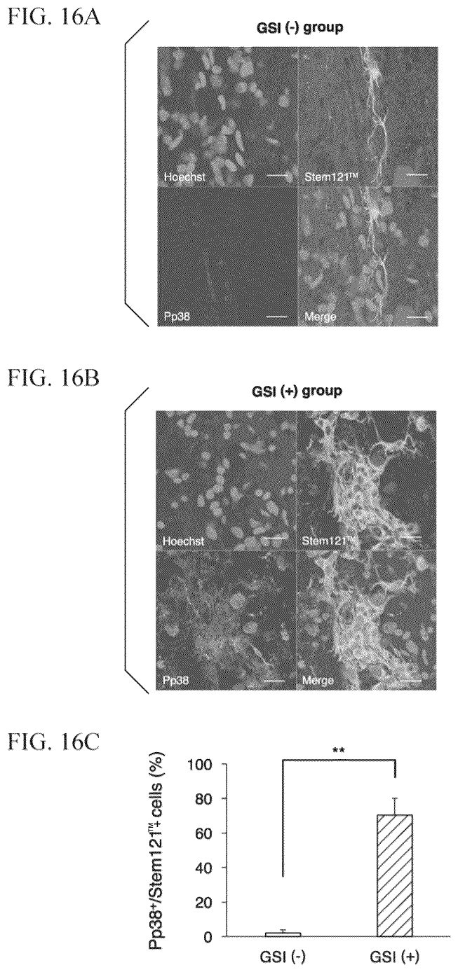

FIG. 16A and FIG. 16B are representative microscopic images showing the results of immunostaining. FIG. 16A shows the results for the GSI (−) group, and FIG. 16B shows the results for the GSI (+) group. In FIG. 16A and FIG. 16B, “Pp38” indicates phosphorylated p38 MAPK, and “Merge” indicates the results of superimposing microscopic images. The scale bar is 20 μm.

As a result, it was found that in the GSI (+) group, STEM121™-positive transplanted cells and a phosphorylated p38-positive region were co-localized.

FIG. 16C is a graph showing the results of calculating the proportion of phosphorylated p38-positive cells in STEM121™-positive transplanted cells based on FIG. 16A and FIG. 16B (n=10). In FIG. 16C, the term “GSI (−)” indicates the results of transplanting non-DAPT-treated human iPS cell-derived neurospheres, and the term “GSI (+)” indicates the results of transplanting DAPT-treated human iPS cell-derived neurospheres. Furthermore, the symbol “**” indicates that there is a significant difference at p<0.01.

As a result, it was found that the proportion of phosphorylated p38-positive cells in the STEM121™-positive transplanted cells was significantly increased in the GSI (+) group as compared to the GSI (−) group.

The above-described results indicate that when human iPS cell-derived neurospheres were treated with a γ-secretase inhibitor, phosphorylation of p38 MAPK is promoted, and this leads to axonal regeneration.

### Experimental Example 5

(Analysis of Mature Neurons Derived from Transplanted Cells)

It has been reported that interventions in the serotonergic activity after spinal cord injury restore motor function through activation of the central pattern generator (CPG). Thus, the effectiveness of transplantation of human iPS cell-derived neurospheres treated with a γ-secretase inhibitor on CPG was examined by immunostaining.

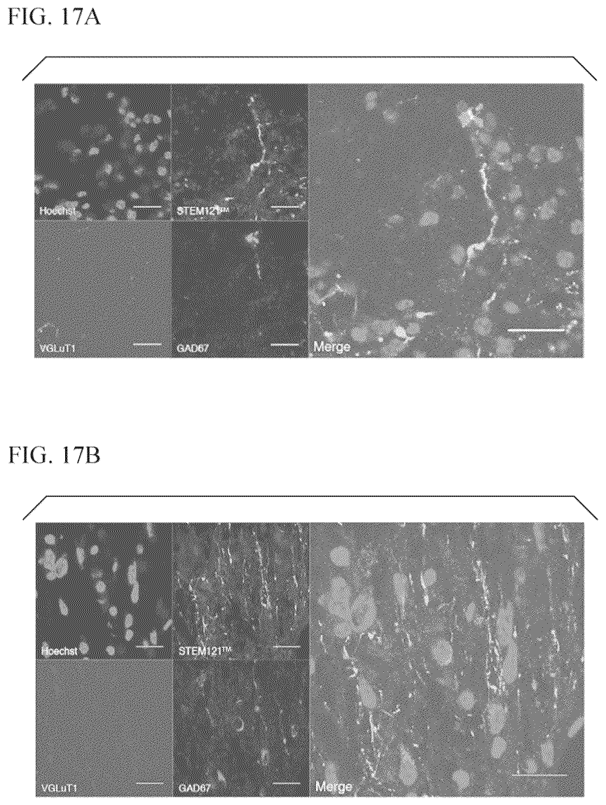

FIG. 17A and FIG. 17B are representative microscopic images showing the results of immunostaining. As a result, STEM121™-positive/vesicular glutamate transporter-1 (VGLuT1)-positive excitatory neurons were almost not observed.

On the other hand, as a result of a quantitative analysis, it was found that 78% of STEM121™-positive cells were glutamic acid decarboxylase 67 (GAD67)-positive inhibitory neurons. This result indicates that the neurons derived from the transplanted cells are GABAergic.

Furthermore, as shown in FIG. 17B, it was found that the STEM121™-positive transplanted cells were connected to the STEM121™-negative/GAD67-positive host mouse cells.

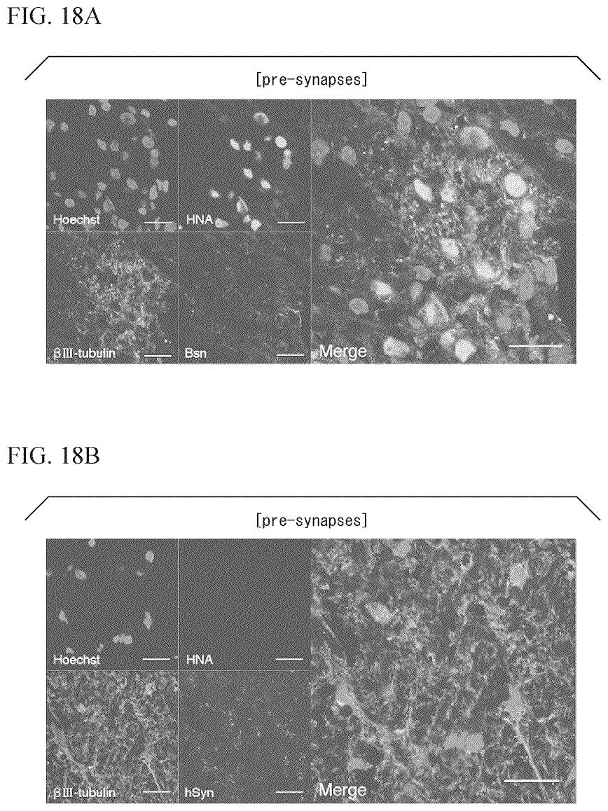

Subsequently, further immunostaining was performed in order to examine whether neurons derived from transplanted cells treated with a γ-secretase inhibitor were incorporated into the neural circuit of the host. FIG. 18A and FIG. 18B are representative microscopic images showing the results of immunostaining.

As a result, βIII-tubulin-positive/human nuclear antigen (HNA)-positive transplanted cells that had been transplanted into the parenchyma were co-localized with the ends of neuronal fibers of a bassoon (Bsn)-positive host, the marker being a mouse-specific pre-synapse marker.

Furthermore, it was found that the ends of synaptophysin (hSyn)-positive neuronal fibers, the marker being a human-specific pre-synaptic marker, are close to the mouse neurons of a βIII-tubulin-positive/human nuclear antigen (HNA)-negative host.

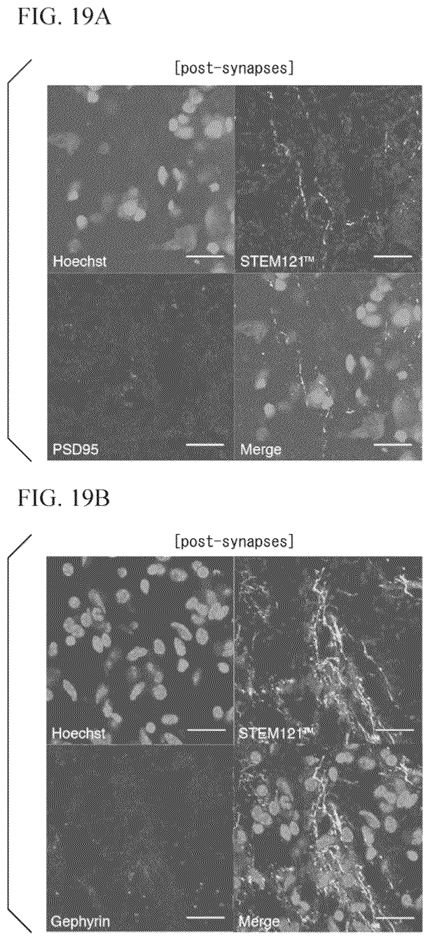

FIG. 19A and FIG. 19B are representative microscopic images showing the results of immunostaining. As a result, it was found that post-synaptic density protein 95 (PSD95)-positive synaptic terminals, the marker being a post-synaptic marker of the terminal of an excitatory synapse, are extremely rare. In addition, it was found that Gephryin-positive synaptic terminals, the marker being a post-synaptic marker of the terminal of an inhibitory synapse, are close to the STEM121™-positive transplanted cells.

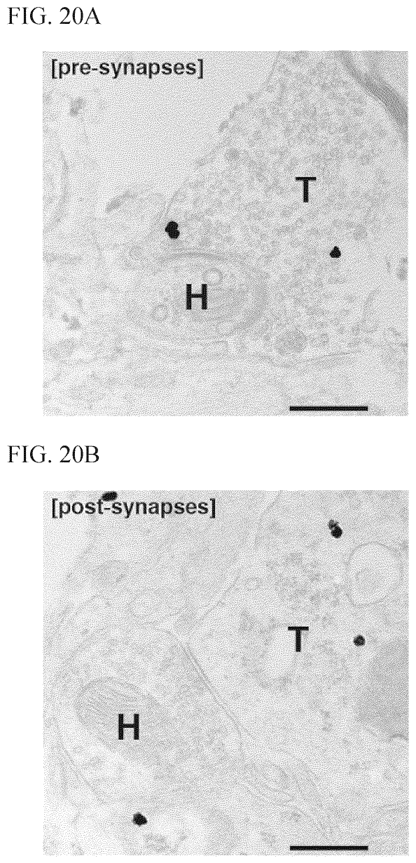

FIG. 20A and FIG. 20B are representative images showing the results of immunoelectron microscopy of synapses formed between transplanted cell-derived human neurons and host mouse neurons. The scale bar is 500 nm. In FIG. 20A and FIG. 20B, “T” indicates a neuron derived from a transplanted cell, and “H” indicates a host neuron. Furthermore, black dots indicate that the cells are STEM121™-positive cells derived from transplanted cells.

As a result, a large number of STEM121™-positive pre-synaptic structures and post-synaptic structures derived from transplanted cells were observed, and synaptic connections between transplanted cell-derived human neurons and host mouse neurons were observed at the site of a spinal cord injury.

### Experimental Example 6

(Regeneration of Reticulospinal Tract by Transplantation of Human iPS Cell-derived Neurospheres Treated with γ-secretase Inhibitor Into Chronic Phase Spinal Cord Injury Model Mice)

The reticulospinal tract (RtST) is a neuronal fiber that descends from the reticular formation and terminates in the spinal cord, and is known to play an important role in initiating spontaneous movement and controlling posture.

In order to assess the regeneration of the reticulospinal tract from the brainstem, 77 days after transplantation of human iPS cell-derived neurospheres into the above-mentioned chronic phase spinal cord injury model mice, neuronal tract tracing was performed by biotinylated dextranamine (BDA) staining.

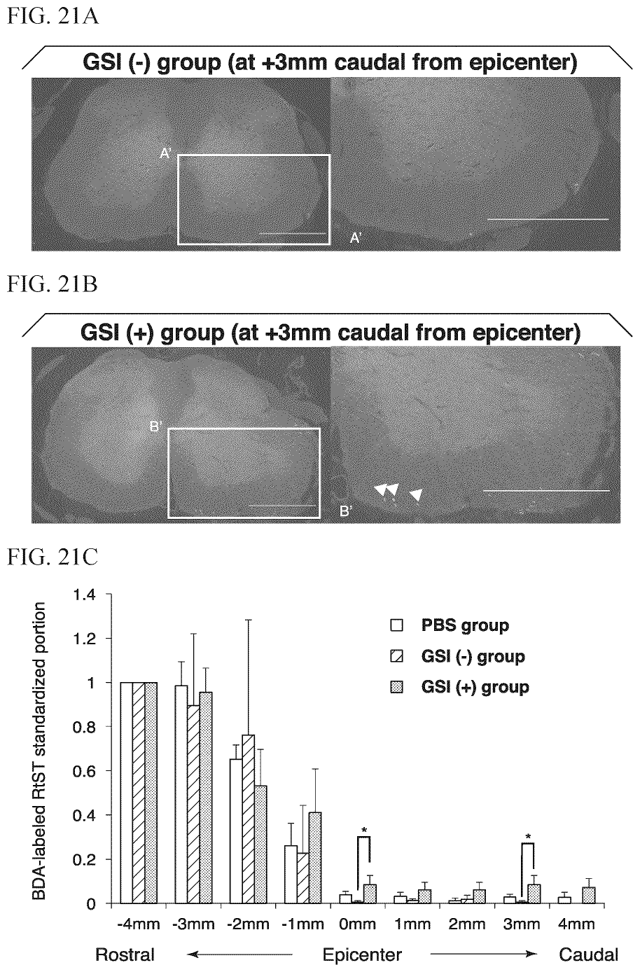

FIG. 21A and FIG. 21B are representative microscopic images of spinal cord sections in which a BDA-stained reticulospinal tract was observed. FIG. 21A is representative images of the GSI (−) group at a position 3 mm away from the site of a spinal cord injury toward the caudal side. The image on the right-hand side is an enlargement of the region surrounded by the square in the image on the left-hand side. The scale bar is 500 μm.

FIG. 21B is representative images of the GSI (+) group at a position 3 mm away from the site of a spinal cord injury toward the caudal side. The image on the right-hand side is an enlargement of the region surrounded by the square in the image on the left-hand side. The scale bar is 500 μm. The arrowheads indicate reticulospinal tract fibers.

FIG. 21C is a graph showing the results of measuring the relative value of the proportion of the area of BDA-stained reticulospinal tract fibers at various positions covering from a position 4 mm away toward the rostral side of the site of a spinal cord injury extending to a position 4 mm away toward the caudal side of the site of a spinal cord injury (n=4). In FIG. 21C, the symbol “*” indicates that there is a significant difference at p<0.05.

As a result, in the GSI (+) group, BDA-labeled reticulospinal tract fibers extending from the rostral side to the caudal side at the site of a spinal cord injury were observed. In addition, it was found that in the GSI (+) group, the proportion of the area of reticulospinal tract fibers was significantly increased at the site of a spinal cord injury and at a position 3 mm away from the site of a spinal cord injury toward the caudal side, as compared to the GSI (−) group and the PBS group.

### Experimental Example 7

(Restoration of Motor Function by Transplantation of Human iPS Cell-derived Neurospheres Treated with γ-secretase Inhibitor Into Chronic Phase Spinal Cord Injury Model Mice)

### <<Evaluation Based on BMS Score>>

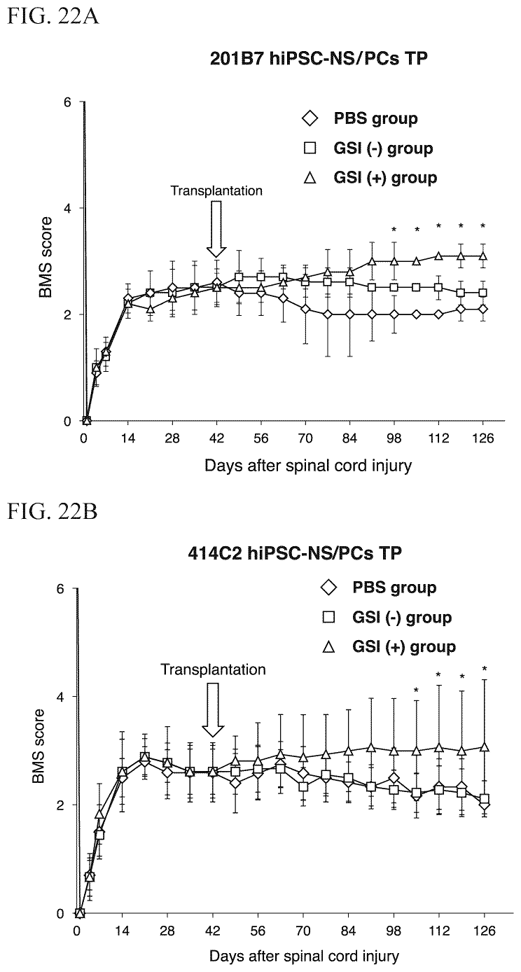

After transplantation of human iPS cell-derived neurospheres into the above-mentioned chronic phase spinal cord injury model mice, the motor function of the mice was evaluated over time. FIG. 22A and FIG. 22B are graphs showing the measurement results of the Basso Mouse Scale (BMS) scores of the mice in various groups (n=10 in each group). FIG. 22A shows the results of transplantation of 201B7 cell-derived neurospheres, and FIG. 22B shows the results of transplantation of 414C2 cell-derived neurospheres.

As a result, significant restoration of motor function was not observed in the GSI (−) group as compared to the PBS group. In contrast, in the GSI (+) group, significant restoration of motor function was observed 56 days after cell transplantation, as compared to the PBS group, and the restored motor function was maintained thereafter.

### <<Evaluation Using Treadmill>>

84 days after transplantation of human iPS cell-derived neurospheres into the above-mentioned chronic phase spinal cord injury model mice, walking function was evaluated using a DigiGait system (manufactured by Mouse Specifics, Inc.).

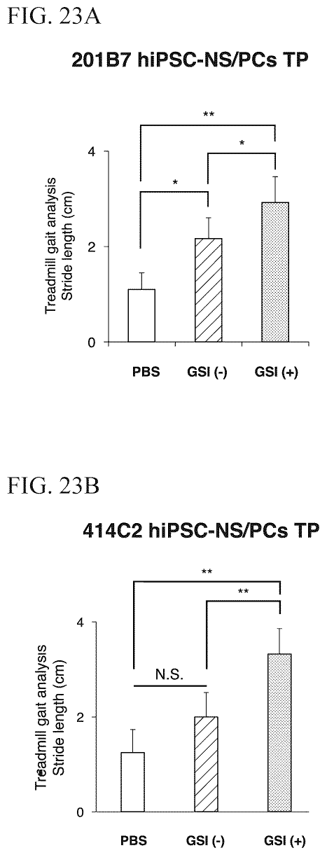

FIG. 23A to FIG. 23D are graphs showing the evaluation results for the walking function. FIG. 23A and FIG. 23C show the results of transplantation of 201B7 cell-derived neurospheres, and FIG. 23B and FIG. 23D show the results of transplantation of 414C2 cell-derived neurospheres (n=10 in each group). Furthermore, FIG. 23A and FIG. 23B show the analysis results for the stride length, and FIG. 23C and FIG. 23D show the analysis results for the stance angle. In FIG. 23A to FIG. 23D, the symbol “**” indicates that there is a significant difference at p<0.01, the symbol “*” indicates that there is a significant difference at p<0.05, and the term “N.S.” indicates that there is no significant difference.

As a result, the mice in the GSI (+) group exhibited significantly long stride lengths and significantly short stance angles as compared to the mice in the GSI (−) group and the PBS group. Furthermore, all the mice in the GSI (+) group walked sufficiently well on the treadmill and walked at a speed of 7 cm/sec.

### <<Evaluation by Rotarod Test>>

84 days after transplantation of human iPS cell-derived neurospheres into the above-mentioned chronic phase spinal cord injury model mice, the motor function was evaluated using a rotarod device (manufactured by Muromachi Kikai Co., Ltd.). The rotarod device is a device having a disk with a diameter of 40 cm at both ends of a plastic rod having a diameter of 3 cm and a length of 8 cm. The rotarod device was rotated at 20 rpm, a mouse was placed on the rod of the device, and the time (seconds) for which the mouse could remain on the rod was measured. The test was performed five times, and the time (seconds) for which the mouse could remain for the longest time was recorded.

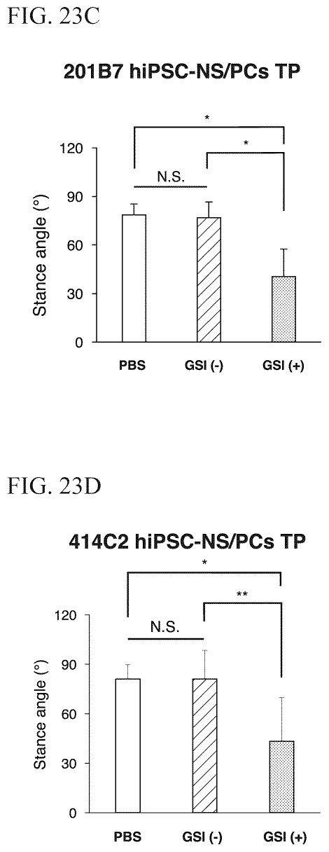

FIG. 24A and FIG. 24B are graphs showing the results of the rotarod test. FIG. 24A shows the results for the transplantation of 201B7 cell-derived neurospheres, and FIG. 24B shows the results for the transplantation of 414C2 cell-derived neurospheres (n=10 in each group). In FIG. 24A and FIG. 24B, the symbol “**” indicates that there is a significant difference at p<0.01, the symbol “*” indicates that there is a significant difference at p<0.05, and the term “N.S.” indicates that there is no significant difference.

As a result, the mice in the GSI (+) group could remain on the rotarod device for significantly long times as compared to the mice in the GSI (−) group and the mice in the PBS group.

### <<Analysis of BMS Score and Proportion of Area of Reticulospinal Tract Fibers>>

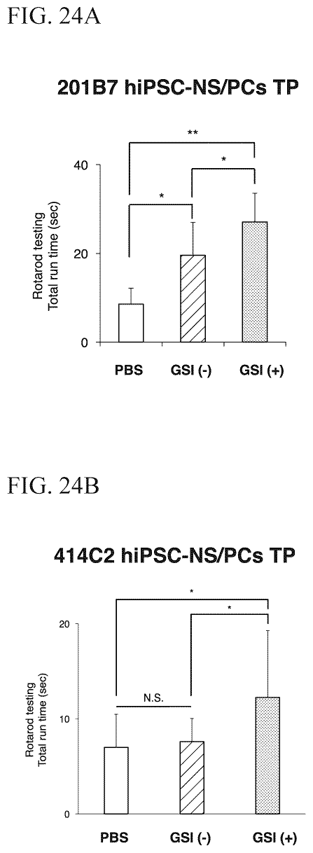

FIG. 25 is a representative graph showing the relationship between the proportion of the area of BDA-labeled reticulospinal tract fibers measured at a position 3 mm away from the site of a spinal cord injury toward the caudal side, and the above-mentioned BMS score (n=14).

As a result, a significant correlation between the two was observed at the site of a spinal cord injury and at the position 3 mm away from the site of a spinal cord injury toward the caudal side and the caudal position. The highest correlation coefficient was obtained at the position 3 mm away from the site of a spinal cord injury toward the caudal side, with R²=0.855.

### Experimental Example 8

(Analysis of Human iPS Cell-derived Neurospheres Treated with γ-secretase Inhibitor—2)

Phosphorylation of p38 MAPK in human iPS cell-derived neurospheres treated with a γ-secretase inhibitor was examined.

Specifically, neurospheres derived from 201B7 cells, which are cells of a human iPS cell strain, were cultured for one day in the presence of a secretase inhibitor at a final concentration of 10 μM. Subsequently, the neurospheres were cultured in a growth factor-free medium in a 95% air environment at 37° C. and 5% CO₂ for 14 days and were differentiated into nerurons. Subsequently, on the 14th day after the initiation of differentiation induction, the cells were fixed with 4% paraformaldehyde in 0.1 M PBS and observed. DAPT was used as the γ-secretase inhibitor. Immunostaining of phosphorylated p38 MAPK and βIII-tubulin was performed. In addition, Hoechst 33342 (CAS Number: 23491-52-3) was used to stain the cell nuclei.

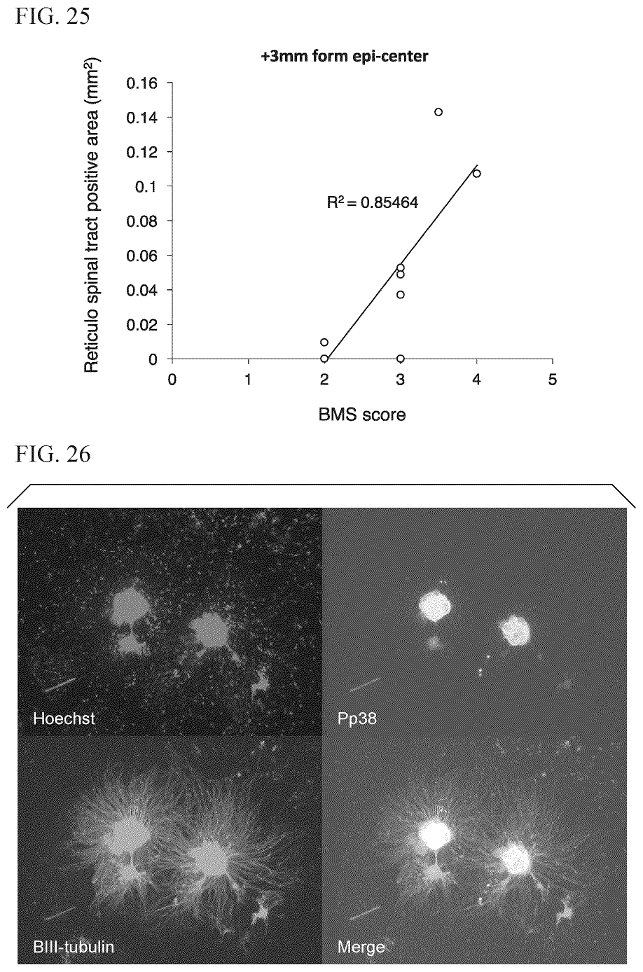

FIG. 26 is representative microscopic images showing the results of immunostaining. In FIG. 26, “Pp38” represents phosphorylated p38 MAPK, and “Merge” indicates the results of superimposing microscopic images. The scale bar is 20 μm.

As a result, it was found that phosphorylation of p38 MAPK was enhanced in the neurons derived from the neurospheres treated with a γ-secretase inhibitor.

According to the present invention, a technology for treating spinal cord injury can be provided.

1-7. (canceled)

8. A method for treating spinal cord injuries, the method comprising transplanting an effective amount of a neurosphere, in which phosphorylation of p38 MAPK is enhanced, into an injured site of a subject suffering from a spinal cord injury.

9. The method of claim 1, wherein the neurosphere is cultured with an effective amount of a γ-secretase inhibitor.

10. The method of claim 1, wherein the neurosphere is cultured with an effective amount of a γ-secretase inhibitor for 20 to 24 hours.

11. The method of claim 2, wherein the γ-secretase inhibitor comprises DAPT or compound 34.

12. The method of claim 2, wherein the γ-secretase inhibitor comprises a concentration of about 10 to 1000 μM.

13. The method of claim 2, wherein the γ-secretase inhibitor comprises a concentration of about 10 μM.

14. The method of claim 1, wherein the neurosphere is obtained from human pluripotent stem cells comprising embryonic stem cells (ES cells) or induced pluripotent stem cells (iPS cells).

15. The method of claim 1, wherein the spinal cord injury is a chronic phase injury.

16. The method of claim 1, wherein the neurosphere is transplanted into an epicenter of the spinal cord injury.

17. A method of treating a chronic-phase spinal cord injury in a subject, comprising:
(a) culturing a pluripotent stem cell-derived neurosphere in the presence of a γ-secretase inhibitor under conditions and for a time sufficient to enhance phosphorylation of p38 MAPK in the neurosphere relative to a control cultured without the inhibitor; and
(b) transplanting the γ-secretase-inhibitor treated neurosphere into an injured spinal cord of a subject, thereby improving motor function of the subject.

18. The method of claim 10, wherein the neurosphere is cultured with an effective amount of a γ-secretase inhibitor for 20 to 24 hours.

19. The method of claim 10, wherein the γ-secretase inhibitor comprises DAPT or compound 34.

20. The method of claim 10, wherein the γ-secretase inhibitor comprises a concentration of about 10 to 1000 μM.

21. The method of claim 10, wherein the neurosphere is obtained from human pluripotent stem cells comprising embryonic stem cells (ES cells) or induced pluripotent stem cells (iPS cells).

22. A method of treating a spinal cord injury in a subject, the method comprising transplanting into an injured spinal cord site an effective amount of a neurosphere in which phosphorylation of p38 MAPK is enhanced compared to a control neurosphere that has not contacted a γ-secretase inhibitor, thereby treating the spinal cord injury.

23. The method of claim 15, wherein the neurosphere is cultured with an effective amount of a γ-secretase inhibitor for 20 to 24 hours.

24. The method of claim 15, wherein the γ-secretase inhibitor comprises DAPT or compound 34.

25. The method of claim 15, wherein the γ-secretase inhibitor comprises a concentration of about 10 to 1000 μM.

26. The method of claim 15, wherein the neurosphere is obtained from human pluripotent stem cells comprising embryonic stem cells (ES cells) or induced pluripotent stem cells (iPS cells).

27. The method of claim 15, wherein the spinal cord injury is a chronic phase injury.
